(19) United States
(12) Patent Application Publication
(10) Pub. No.: US 2024/0287893 A1
(43) Pub. Date: Aug. 29, 2024

(54) METHODS AND APPARATUS FOR REALTIME INLINE FRACTURING FLUIDS MONITORING

(71) Applicant: SAUDI ARABIAN OIL COMPANY, Dhahran (SA)

(72) Inventors: Qasim Sahu, Jeddah-Thawul (SA); Adrian Cesar Cavazos Sepulveda, Nuevo Leon (MX)

(73) Assignee: SAUDI ARABIAN OIL COMPANY, Dhahran (SA)

(21) Appl. No.: 18/176,227

(22) Filed: Feb. 28, 2023

(51) Int. Cl. E21B 47/107; E21B 43/26; G01N 33/28

(52) U.S. Cl. CPC E21B 47/107; E21B 43/26; E21B 43/2607; G01N 21/3577; G01N 33/2823; E21B 2200/20; G01N 2201/129

(57) ABSTRACT

A monitoring system includes a spectroscopic probe in fluid connection with a fracturing fluid, reference spectra obtained from one or more fracturing fluid components, and a computer in communication with the spectroscopic probe. The computer includes a predictive model generated from the reference spectra. The computer also includes instructions to compare spectroscopic data obtained from the spectroscopic probe with the predictive model. A modified well fracturing system includes a monitoring system located at a surface location of a hydrocarbon-bearing reservoir and a surface pump in fluid connection with the fracturing fluid source and a wellbore of the hydrocarbon-bearing reservoir. A method for monitoring a fracturing fluid is also described.

### BACKGROUND

Hydraulic fracturing is used in the oil and gas industry to stimulate production in hydrocarbon-containing formations. The fracturing is created after drilling a well by injecting suitable fluids such as water or chemicals into the well under pressure to induce fractures in a formation. A variety of fluids has been developed to withstand the high pump rates, shear stresses, and high temperatures and pressures a fracturing fluid may be exposed to. For example, hydraulic fracturing fluids may be aqueous-based gels, emulsions, or foams.

Hydraulic fracturing fluids may also contain proppants including solid proppants such as sand (“frac sand”) or ceramic beads to hold open fractures created in the formation. In such hydraulic fracturing fluids, complex chemical mixtures having sufficient viscosity properties may be included to generate fracture geometry in the formation rock and transport solid proppants holding the fracture open. In this context, the viscosity of the hydraulic fracturing fluids may impact the fracture initiation, propagation and resulting dimensions.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a monitoring system that includes a spectroscopic probe in fluid connection with a fracturing fluid, reference spectra obtained from one or more fracturing fluid components, and a computer in communication with the spectroscopic probe. The computer includes a predictive model generated from the reference spectra. The computer also includes instructions to compare spectroscopic data obtained from the spectroscopic probe with the predictive model.

In another aspect, embodiments disclosed herein relate to a modified well fracturing system including a monitoring system located at a surface location of a hydrocarbon-bearing reservoir and a surface pump in fluid connection with a fracturing fluid source and a wellbore of the hydrocarbon-bearing reservoir.

In another aspect, embodiments disclosed herein relate to a method for monitoring a fracturing fluid that includes obtaining reference spectra for one or more fracturing fluid components to generate one or more data sets, building a predictive model with the one or more data sets to assign rheological properties to different fracturing fluid compositions, installing a monitoring system on fracturing equipment to provide a modified well fracturing system, injecting the fracturing fluid through the modified well fracturing system, and collecting spectral data of the fracturing fluid.

Other aspects and advantages of the claimed subject matter will be apparent from the following description and the appended claims.

### BRIEF DESCRIPTION OF DRAWINGS

FIG. 1 is a schematic of a monitoring system in accordance with one or more embodiments.

FIG. 2 is a computer in accordance with one or more embodiments.

FIG. 3 is a schematic of a modified well fracturing system accordance with one or more embodiments.

FIG. 4 is a flow diagram of a method of monitoring a fracturing fluid accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Fracturing is a method used in the oil and gas industry for increasing the recovery from tight and/or unconventional reservoirs. These types of reservoirs will not flow neutrally because of extremely low hydraulic permeability within the reservoir. Hence, a stimulation treatment, such as hydraulic fracturing, is often required to extract hydrocarbons from the reservoir. The hydraulic fracturing treatment involves injecting multiple fluids in a single pumping operation at a target location of the reservoir. Crack propagation via fracturing fluid injection generates a highly conductive channel that connects the portions of the reservoir at increased depths with a wellbore, which allows for the improvement in hydrocarbon production to the surface of the reservoir. Thus, there remains a need to develop methods and systems to monitor and control fracturing fluids.

Fracturing fluids play a critical role during fracturing processes of a hydrocarbon bearing reservoir with low permeability. Fracturing fluid injection is performed at a pressure greater than the rock fracturing pressure, which is critical to initiating and propagating a crack in the target location. For instance, the objective of the fracturing fluids is often to create enough pressure to initiate and propagate the desired reservoir fracture, transport solid particles, such as proppant, downhole, and control fluid loss inside the fractured reservoir. In effect, it is critical to measure and monitor properties of the fracturing fluids, such as rheological properties including viscosity.

Embodiments in accordance with the present disclosure generally relate to a system and methods for monitoring one or more properties of a fracturing fluid. Monitored properties may include viscosity of the fluid, additive concentration, fluid density, among others. As one of ordinary skill may appreciate, viscosity is a measure of the resistance of a fluid which is being deformed by either shear stress or tensile stress. Viscosity describes a fluid's internal resistance to flow and may be thought of as a measure of fluid friction.

In one or more embodiments, one or more monitored properties are correlated as a function of data obtained from spectroscopic measurements of a fracturing fluid and the density of the crude oil sample. The spectroscopic measurements may be collected in a static (“batch”) sample or in a continuous flow system, such as a fracturing fluid transport line.

### Monitoring System

In one aspect, embodiments disclosed herein relate to a monitoring system for assigning a property to a fracturing fluid. The monitoring system of one or more embodiments includes a spectroscopic probe, a fracturing fluid, and a computer. In one or more embodiments, the spectroscopic probe is in fluid connection with the fracturing fluid, such that spectroscopic measurements are taken from a single sample of a fracturing fluid or a continuously flowed fracturing fluid. In such embodiments, the monitoring system may be an in-line measurement system configured to measure a continuously flowed fracturing fluid.

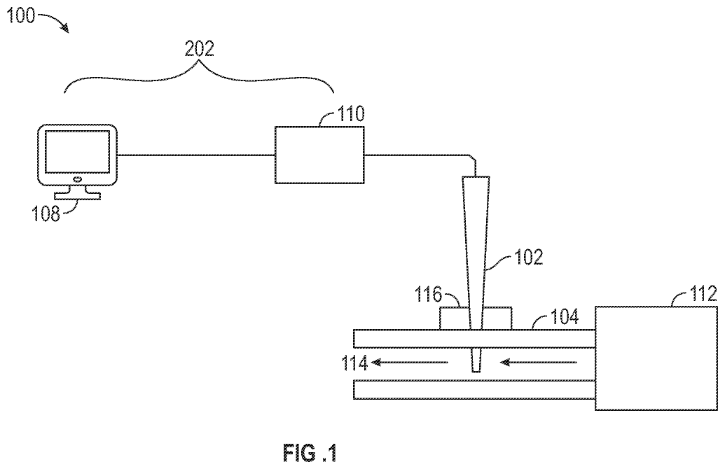

FIG. 1 is a schematic of a monitoring system in accordance with one or more embodiments. As shown in FIG. 1, a monitoring system 100 includes a spectroscopic probe 102 in fluid connection with a fracturing fluid 114. The spectroscopic probe 102 is installed on a fluid transport line 104 with connection 116. Connection 116 may include a connecting device selected from, for example, a screw connection, a plug connection, or both. In FIG. 1, the fracturing fluid 114 is continuously flowed from a fracturing fluid source 112 via the fluid transport line 104 to a target location. As such, spectroscopic probe 102 is configured to collect a series of spectroscopic measurements of the continuously flowed fracturing fluid.

The monitoring system 100 includes computer 202 in communication with the spectroscopic probe 102. In FIG. 1, computer 202 includes a data processor 110, an interface 108, among other components as further described below. The computer 202 of FIG. 1 is configured to collect spectroscopic measurements from spectroscopic probe 102, process the collected spectroscopic measurements, store one or more data sets generated from the spectroscopic measurements, derive one or more fluid properties from the data sets, or combinations thereof. The spectroscopic measurements may include reference spectra or

As one of ordinary skill may appreciate, the fracturing fluid may be formulated to include components selected from the group consisting of one or more crosslinkers, a polymeric gelling agent, a base fluid, one or more acids, among other additives. The additives may be any conventionally known and one of ordinary skill in the art will, with the benefit of this disclosure, appreciate that the selection of said additives will be dependent upon the intended application of the fracturing fluid. In some embodiments, the additives may be one or more selected from viscoelastic surfactants, clay stabilizers, scale inhibitors, corrosion inhibitors, biocides, friction reducers, thickeners, fluid loss additives, and the like.

As mentioned above, the monitoring system is configured to collect one or more spectroscopic measurements from the fracturing fluid 114 with the spectroscopic probe 102. The monitoring system 100 may be configured to collect a series of spectroscopic measurements, such as a series of measurements in a continuously flowing fluid. The spectroscopic probe may be a probe that measures spectroscopic data from a sample, such as a fracturing fluid or a reference fluid sample, with infrared energy in a designated wavelength range.

The spectroscopic probe 102 may be configured to collect spectroscopic data selected from the group consisting of Fourier Transform Infrared Spectroscopic (FTIR) data, a Dual Comb Spectroscopic (DCS) data, Multi Comb Spectroscopic data, wavelength specific data, or combinations thereof. In one or more embodiments, the wavelength specific data may include spectroscopic measurements in an infrared wavelength range selected from the group consisting of a near infrared (NIR) wavelength range, a mid-infrared (MIR) wavelength range, a far infrared (FIR) wavelength range, or combinations thereof. Moreover, these techniques described above can be used in absorption, reflection, transmission, diffuse reflectance, and/or transfections measurements.

As one of ordinary skill may appreciate, infrared energy is the electromagnetic energy of molecular vibration. The energy band is defined for convenience as the near infrared (0.78-2.50 microns), the infrared (or mid-infrared) 2.50-40.0 microns, and the far infrared (40.0-1000 microns). However, even though official standards, textbooks, and the scientific literature generally state that the NIR spectral region extends from 780-2500 nanometers (12821-4000 cm⁻¹), a simple set of liquid phase hydrocarbon spectra demonstrates that the vibrational information characterized by the harmonic vibrations of the C—H stretch fundamental and their corresponding combination bands occurs from approximately 690-3000 nm. The predominant near-infrared spectral features include: the methyl C—H stretching vibrations, methylene C—H stretching vibrations, aromatic C—H stretching vibrations, and O—H stretching vibrations. Minor but still important spectral features include: methoxy C—H stretching, carbonyl associated C—H stretching; N—H from primary amides, secondary amides (both alkyl, and aryl group associations), N—H from primary, secondary, and tertiary amines, and N—H from amine salts. Near infrared spectroscopy is used where multicomponent molecular vibrational analysis is required in the presence of interfering substances.

In one or more embodiments, the spectroscopic probe 102 is a near-infrared spectroscopic probe. The near infrared spectra consist of overtones and combination bands of the fundamental molecular absorptions found in the mid infrared region. Near infrared spectra consist of generally overlapping vibrational bands that may appear non-specific and poorly resolved. The use of a machine learning algorithm, chemometric mathematical data processing and multiple harmonics can be used to calibrate for qualitative and quantitative analysis despite these apparent spectroscopic limitations.

Note that a near infrared spectrum consists in the convolution of the measuring instrument function with the unique optical characteristics of the sample being measured (i.e., the sample is an active optical element of the spectrometer). The reference values are those chemical or physical parameters to be predicted using the NIR spectroscopic measurements. A spectrum may contain information related to the sample chemistry measured using any specific reference method. Spectra-structure correlation provides a basis for the establishment of a known cause and effect relationship between instrument response and reference (analyte) data, in order to provide a more scientific basis for chemometric-based near infrared spectroscopy.

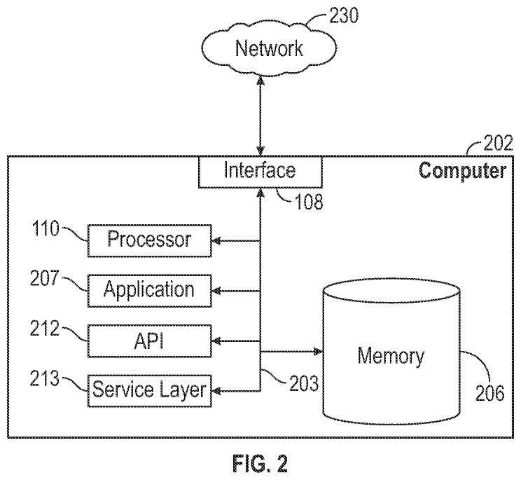

FIG. 2 is a computer 202 in accordance with one or more embodiments. As such, computer 202 is used to provide computational functionalities associated with described chemometric analyses, machine learning algorithms, methods, functions, processes, flows, and procedures as described in this disclosure, according to one or more embodiments. The computer 202 may correlate one or more properties of the fracturing fluid with spectroscopic data collected from the spectroscopic probe. One or more properties of a fracturing fluid may include a viscosity of the fluid, an additive concentration, a density of the fluid, or combinations thereof.

As shown in FIG. 2, the illustrated computer 202 is intended to encompass any computer such as a server, desktop computer, laptop/notebook computer, wireless data port, smart phone, personal data assistant (PDA), tablet computer, one or more processors within these devices, or any other suitable processing device, including both physical or virtual instances (or both) of the computer. Additionally, the computer 202 may include a computer that includes an input device, such as a keypad, keyboard, touch screen, or other device that can accept user information, and an output device that conveys information associated with the operation of the computer 202, including digital data, visual, or audio information (or a combination of information), or a GUI.

The computer 202 can serve in a role as a client, network component, a server, a database or other persistency, or any other component (or a combination of roles) of a computer for performing the subject matter described in the instant disclosure. The illustrated computer 202 is communicably coupled with a network 230. In some implementations, one or more components of the computer 202 may be configured to operate within environments, including cloud-computing-based, local, global, or other environment (or a combination of environments).

At a high level, the computer 202 is an electronic computer operable to receive, transmit, process, store, or manage data and information associated with the described subject matter. The calculated and assigned results in accordance with the systems and methods herein are displayed, audibly outputted, printed, and/or stored to memory for use as described herein. According to some implementations, the computer 202 may also include or be communicably coupled with an application server, e-mail server, web server, caching server, streaming data server, business intelligence (BI) server, or other server (or a combination of servers).

The computer 202 can receive requests over network 230 from a client application (for example, executing on another computer and responding to the received requests by processing the said requests in an appropriate software application. In addition, requests may also be sent to the computer 202 from internal users (for example, from a command console or by other appropriate access method), external or third-parties, other automated applications, as well as any other appropriate entities, individuals, systems, or computers.

Each of the components of the computer 202 can communicate using a system bus 203. In some implementations, any or all of the components of the computer 202, both hardware or software (or a combination of hardware and software), may interface with each other or the interface 108 (or a combination of both) over the system bus 203 using an application programming interface (API) 212 or a service layer 213 (or a combination of the API 212 and service layer 213. The API 212 may include specifications for routines, data structures, and object classes. The API 212 may be either computer-language independent or dependent and refer to a complete interface, a single function, or even a set of APIs. The service layer 213 provides software services to the computer 202 or other components (whether or not illustrated) that are communicably coupled to the computer 202. The functionality of the computer 202 may be accessible for all service consumers using this service layer. Software services, such as those provided by the service layer 213, provide reusable, defined functionalities through a defined interface. For example, the interface may be software written in JAVA, C++, or other suitable language providing data in extensible markup language (XML) format or another suitable format. While illustrated as an integrated component of the computer 202, alternative implementations may illustrate the API 212 or the service layer 213 as stand-alone components in relation to other components of the computer 202 or other components (whether or not illustrated) that are communicably coupled to the computer 202. Moreover, any or all parts of the API 212 or the service layer 213 may be implemented as child or sub-modules of another software module, enterprise application, or hardware module without departing from the scope of this disclosure.

The computer 202 includes an interface 108. Although illustrated as a single interface 108 in FIG. 2, two or more interfaces 108 may be used according to particular needs, desires, or particular implementations of the computer 202. The interface 108 is used by the computer 202 for communicating with other systems in a distributed environment that are connected to the network 230. Generally, the interface 108 includes logic encoded in software or hardware (or a combination of software and hardware) and operable to communicate with the network 230. More specifically, the interface 108 may include software supporting one or more communication protocols associated with communications such that the network 230 or interface's hardware is operable to communicate physical signals within and outside of the illustrated computer 202.

The computer 202 includes at least one computer system. Although illustrated as a single computer system in FIG. 2, two or more processors may be used according to particular needs, desires, or particular implementations of the computer 202. Generally, the computer system executes instructions and manipulates data to perform the operations of the computer 202 and any chemometric analyses, machine learning algorithms, methods, functions, processes, flows, and procedures as described in the instant disclosure.

The computer 202 also includes a memory 206 that holds data for the computer 202 or other components (or a combination of both) that can be connected to the network 230. For example, memory 206 can be a database storing data consistent with this disclosure. Although illustrated as a single memory 206 in FIG. 2, two or more memories may be used according to particular needs, desires, or particular implementations of the computer (202) and the described functionality. While memory 206 is illustrated as an integral component of the computer 202, in alternative implementations, memory 206 can be external to the computer 202. In one or more embodiments, the computer includes data storage memory configured to store data used and/or generated by the one or more modules of the present invention, including viscosity of reference fracturing fluids, reference spectra, collected infrared spectroscopy data, or portions thereof used by the one or more modules of the present system, and calculated indicative properties generated by the one or more modules of the present system.

The application 207 is an algorithmic software engine providing functionality according to particular needs, desires, or particular implementations of the computer 202, particularly with respect to functionality described in this disclosure. For example, application 207 can serve as one or more components, modules, applications, etc. Further, although illustrated as a single application 207, the application 207 may be implemented as multiple applications 207 on the computer 202. In addition, although illustrated as integral to the computer 202, in alternative implementations, the application 207 can be external to the computer 202.

There may be any number of computers 202 associated with, or external to, a computer system containing computer 202, wherein each computer 202 communicates over network 230. Further, the term “client,” “user,” and other appropriate terminology may be used interchangeably as appropriate without departing from the scope of this disclosure. Moreover, this disclosure contemplates that many users may use one computer 202, or that one user may use multiple computers 202.

### Modified Well Fracturing System

In another aspect, one or more embodiments of the present disclosure are directed to a modified well fracturing system. The modified well fracturing includes a monitoring system as described above installed at a surface location of a hydrocarbon bearing reservoir. The modified well fracturing may include a well fracturing system made of fracturing equipment and other components configured for reservoir fracturing as known to those skilled in the art.

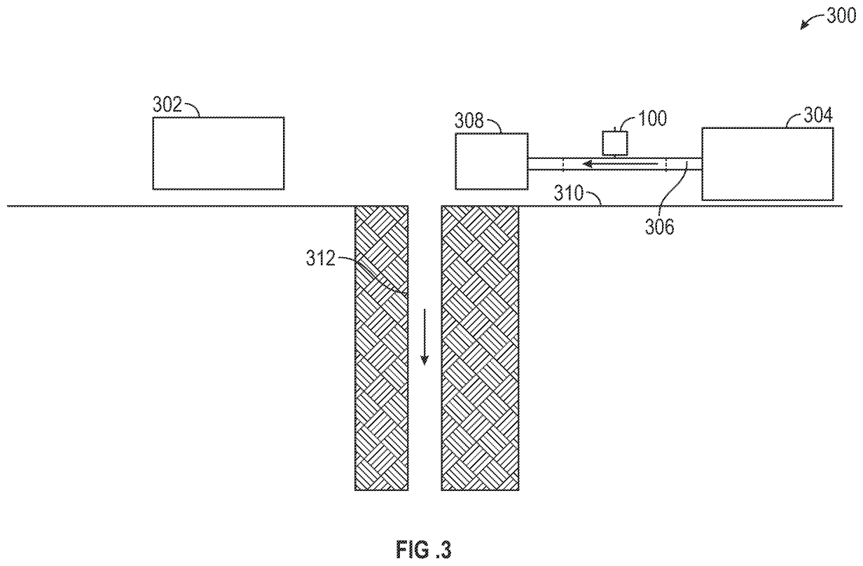

FIG. 3 is a schematic of a modified well fracturing system 300 in accordance with one or more embodiments. As shown in FIG. 3, monitoring system 100 is installed in a well fracturing system at a surface location 310 of a hydrocarbon bearing reservoir. The well fracturing system may generally include, for example, a fracturing fluid source 304, one or more fracturing fluid transport line 306 fluidly connected to the fracturing fluid source 304, one or more surface pumps 308 (e.g., frac pumps, which may be provided on trucks or trailers) for pumping the fracturing fluid delivered from fracturing fluid transport line(s) 306 downhole at high pressures, and downhole equipment (e.g., casing, piping, etc.) installed in a wellbore 312 to the reservoir for delivering the high pressure fracturing fluid to a selected fracturing zone along the well. The surface pump(s) 308 are in fluid connection with the wellbore 312 of the reservoir such that the surface pump(s) 308 deliver the fracturing fluid into the wellbore 312.

According to embodiments of the present disclosure, fracturing fluid may be mixed on site to provide the fracturing fluid source 304 from fracturing fluid component sources, e.g., including fluid tanks, proppant supply tanks, etc. Additionally, one or more valves (e.g., positioned on component source tanks and/or on flow lines between component source tanks and other equipment) may be used to control relative amounts of the fracturing fluid components provided from the fracturing fluid component sources to be mixed together using, e.g., mixers or blenders. As one of ordinary skill may appreciate, one or more additional components 302 for treating the reservoir may also be included at the surface location 310 of the hydrocarbon bearing reservoir.

The monitoring system 100 may include a spectroscopic probe that may be positioned in the well fracturing system to contact fracturing fluid flowing through the well fracturing system. For example, as shown in FIG. 3, the monitoring system 100 is installed such that the spectroscopic probe is installed on a fracturing fluid transport line 306. In such embodiments, the spectroscopic probe may contact the fracturing fluid after it is mixed and provided from the fracturing fluid source 304 and before the fracturing fluid is pressurized by the surface pump(s) 308. In some embodiments, a spectroscopic probe in a monitoring system 100 may be connected to one or more other fracturing equipment in a well fracturing system such that the spectroscopic probe may contact the fracturing fluid in the well fracturing system.

As described above, the monitoring system 100 may further include a computer in communication with the spectroscopic probe, such that fracturing fluid data collected from the spectroscopic probe may be sent to and processed by the computer system. Once processed by the computer system, the fracturing fluid data collected from the spectroscopic probe may be compared with a predictive model generated for the fracturing fluid. In some embodiments, when the fracturing fluid data collected from the spectroscopic probe does not correspond with the fracturing fluid predictive model, the computer system may be used to control one or more fracturing equipment components in the well fracturing system to alter the fracturing fluid. For example, in some embodiments, the computer system may be in communication with one or more valves to fracturing fluid component sources. In such embodiments, the computer system may generate and execute commands to control (e.g., open, close, or partially open) one or more valves to fracturing fluid component sources to alter the fracturing fluid composition.

### Method of Monitoring a Fracturing Fluid

In another aspect, embodiments of the present disclosure are directed to methods for monitoring a fracturing fluid. The methods of one or more embodiments of the present disclosure may be used during treatment of a hydrocarbon bearing reservoir.

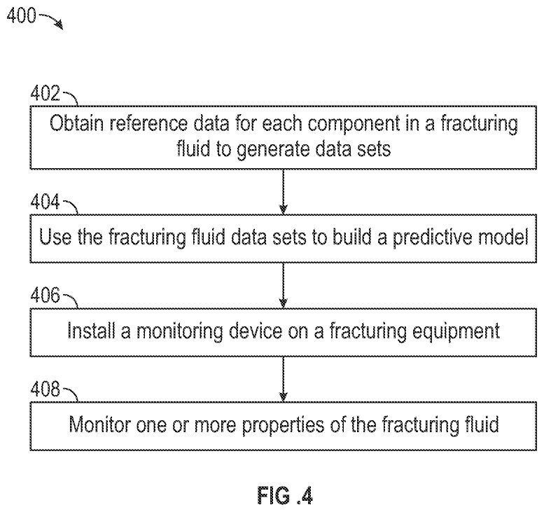

For example, a method 400 of monitoring a fracturing fluid according to embodiments of the present disclosure is shown in FIG. 4. As shown in FIG. 4, the method 400 includes obtaining reference data for each component in a fracturing fluid to generate fracturing fluid data sets 402. The step of obtaining reference data may include collecting reference spectra of one or more fracturing fluid components with the monitoring system as described above. In one or more embodiments, the reference spectra are measured and collected in a laboratory setting using a spectroscopic probe (e.g., an infrared probe) or any equivalent device. In some embodiments, collecting reference spectra of one or more fracturing fluid components may include preparing a reference fracturing fluid including a fracturing fluid component of a known (or predetermined) concentration, mixing the reference fracturing fluid, collecting spectroscopic data (e.g., reference spectra) of the reference fracturing fluid, collecting rheological measurements of the reference fluid, or combinations thereof.

The reference spectra may include spectroscopic measurements collected from one or more reference fracturing fluids. The one or more reference fracturing fluids may include a known concentration of one or more components. In such embodiments, the one or more reference fluids have known rheological properties. For example, the reference spectra may include spectroscopic measurements for all components of a fracturing fluid. The reference spectra may include spectroscopic measurements collected for a plurality of concentrations of each component of the reference fracturing fluid.

In one or more embodiments, a minimum of 250 spectroscopic measurements are needed to generate the datasets. In one or more embodiments, a minimum of 500 spectroscopic measurements are needed to generate the datasets. In one or more embodiments, a minimum of 1000 spectroscopic measurements are needed to generate the datasets. In one or more embodiments, a minimum of 2000 spectroscopic measurements are needed to generate the datasets. In one or more embodiments, a minimum of 3000 spectroscopic measurements are needed to generate the datasets. In some embodiments, spectral data measurements and collection are performed off-site from a well location, such as in a laboratory setting. In some embodiments, spectral data measurements and collection are performed onsite at a well site during a fracturing treatment. In such embodiments, the spectral data measurements collected at the well site can be used to update the model.

In some embodiments, reference spectra is measured and collected for each fracturing fluid component. The method of one or more embodiments may include preparing a component reference fluid from a fracturing fluid component and a base fluid. Fracturing fluid components include, but are not limited to, a polymer agent, crosslinker, acid, clay stabilizer, scale inhibitor, among other fracturing fluid additives. Each fracturing fluid component may be added in a predetermined concentration to a base fluid to form a component reference fluid. For example, a polymer agent reference fluid may be formed by adding a predetermined amount of a polymer agent to a base fluid. A predetermined concentration of an acid may be added to a base fluid to form an acid reference fluid. A predetermined concentration of a clay stabilizer may be added to a base fluid to form a clay stabilizer reference fluid. A predetermined concentration of a scale inhibitor may be added to form a scale inhibitor reference fluid. A predetermined concentration of a crosslinker may be added to a base fluid to form a crosslinker reference fluid.

The method of one or more embodiments may include preparing a plurality of component reference fluids. The plurality of component reference fluids may include a range of fracturing fluid component concentrations added to a base fluid. Each reference fluid of the plurality of component reference fluids may individually include a predetermined concentration of a fracturing fluid component added to a base fluid. The fracturing fluid components may be as described above. In one or more embodiments, the plurality of component reference fluids include a plurality of crosslinker reference fluids, a plurality of polymer agent reference fluids, a plurality of acid reference fluids, a plurality of scale inhibitor reference fluids, a plurality of clay stabilizer reference fluids, or combinations thereof.

In one or more embodiments, the predetermined concentration of a fracturing fluid component in a component reference fluid can be an in a range of 0.01 gallon per thousand gallons (gpt) to 30 gpt. For example, the component reference fluid may include the a fracturing fluid component in an amount ranging from a lower limit of one of 0.01, 0.05, 0.10, 0.25, 0.5, 1, 2, 5, 7.5 and 10 gpt to an upper limit of one of 1, 2.5, 5, 7.5, 10, 11, 12.5, 15, 17.5, 20, 25, and 30 gpt, where any lower limit may be paired with any mathematically compatible upper limit.

In some embodiments, reference spectra is measured and collected for each fracturing fluid component in a plurality of component reference fluids to generate datasets. The datasets include, but are not limited to, high-resolution Fourier transform spectrograms, diffuse reflectance, sonic velocities, or combinations thereof. Once the datasets are generated, a predictive model can be built based on machine learning and/or multivariate analysis. The model can then be used in the field to measure the viscosity inline.

In one or more embodiments, the collected reference data, the collected spectral data of monitored fracturing fluids as described below, or both includes collected data measured from techniques selected from the group consisting of Fourier Transform Infrared Spectroscopy (FTIR), a Dual Comb Spectroscopy (DCS), Multi Comb Spectroscopy, wavelength specific spectroscopy, or combinations thereof.

The method 400 may include generating one or more data sets from the collected reference spectra of the reference fracturing fluids with the computer of the monitoring system. In one or more embodiments, the generated datasets include measured rheological properties for one or more reference fluids as described above. A predictive model may then be generated by using the computer to process the one or more generated data sets 404. In one or more embodiments, the computer of the monitoring system generates a predictive model from chemometric analysis of the one or more data sets with the computer, the computer of the monitoring system uses the data sets obtained from the fracturing fluids to generate a predictive model using machine learning, or both. In one or more embodiments, the predictive model generates predicted rheological properties for different compositions of fracturing fluids.

Machine-learned model types may include, but are not limited to, generalized linear models, Bayesian regression, random forests, and deep models such as neural networks, convolutional neural networks, and recurrent neural networks. Machine-learned model types, whether they are considered deep or not, are usually associated with additional “hyperparameters” which further describe the model. For example, hyperparameters providing further detail about a neural network may include, but are not limited to, the number of layers in the neural network, choice of activation functions, inclusion of batch normalization layers, and regularization strength. It is noted that in the context of machine learning (ML), the regularization of a machine-learned model refers to a penalty applied to the loss function of the machine-learned model and should not be confused with the regularization of a dataset. Commonly, in the literature, the selection of hyperparameters surrounding a machine-learned model is referred to as selecting the model “architecture”. Once a machine-learned model type and hyperparameters have been selected, the machine-learned model is trained to perform a task.

In accordance with one or more embodiments, a machine-learned model type and associated architecture are selected, the machine-learned model is trained to boost the bandwidth of a given dataset, the performance of the machine-learned model is evaluated, and the machine-learned model is used in a production setting (also known as deployment of the machine-learned model).

In one or more embodiments, qualitative and quantitative infrared (IR) spectroscopic methods require the application of chemometric calibration algorithms and statistical methods (i.e., chemometrics) to model IR spectral response to chemical or physical properties (e.g., rheology) of the samples used for data set generation (or “calibration”). In some embodiments, the predictive model includes a partial least square regression model. In some embodiments, the predictive model is a partial least square regression model.

The NIR method of one or more embodiments may rely on the spectra-structure correlations existing between a measured spectral response caused by the harmonics of the fundamental vibrations occurring at infrared frequencies. These harmonic vibrations occur at unique frequencies depending upon the quantity of absorber (analyte), type of absorbing molecules present within the sample, and the sample thickness. Quantitative predictive methods are possible where changes in the response of the near infrared spectrometer are proportional to changes in the concentration of chemical components, or in the physical characteristics (scattering/absorptive properties) of samples undergoing analysis.

When performing multivariate calibrations, analytically valid calibration models require a relationship between X (the instrument response data or spectrum), and Y (the reference data). The use of probability alone tells us only if X and Y “appear” to be related. If no cause-effect relationship exists between spectra-structure correlation and reference values, the model will have no true predictive importance. Thus, knowledge of cause and effect creates a basis for scientific decision-making.

Factors affecting the integrity of the teaching samples (e.g., data sets generated from reference samples) used to calibrate spectrophotometers for individual infrared (e.g., NIR) applications according to one or more embodiments include the variations in sample chemistry, the physical condition of samples, and the measurement conditions. Teaching samples must represent several sample “spaces” to include: compositional space, instrument space, and measurement condition (sample handling and presentation) space.

As one of ordinary skill may appreciate, the number of reference measurements, quality of reference measurements, and the type of predictive model used may affect the ability of the machine learning algorithm to provide accurate predictions of fluid properties. In some embodiments, dataset generation may require pre-processing of the reference measurements (e.g., reference spectra and/or reference rheological measurements) prior to generation of the predictive model. The pre-processing of the reference measurements may require one or more mathematical transformations to remove the effects of light scattering, to highlight peaks, to eliminate multiplicative and baseline shift noise, or combinations thereof.

The method 400 of one or more embodiments includes installing a monitoring system at a surface location of the hydrocarbon bearing reservoir 406 to provide a reservoir fracturing system. Installing the monitoring system may include installing the spectroscopic probe of the monitoring system on a fluid transport line or other equipment of a well fracturing system as described above. The fluid transport line may be in fluid connection with a fracturing fluid source and a wellbore as described above. The fracturing fluid may flow through a modified well fracturing system into a hydrocarbon-bearing reservoir with an injection well.

The method of one or more embodiments may include verifying the accuracy of the predictive model developed from the generated datasets. The predictive model may be verified prior to installation of the monitoring system at a surface location of the hydrocarbon bearing reservoir, after installation of the monitoring system at a surface location of the hydrocarbon bearing reservoir, or both. In such embodiments, the accuracy of the predictive model is verified by obtaining a sample of a fracturing fluid from a well site and measuring the viscosity of the sample. The viscosity of the sample may be measured, for example, on a benchtop viscometer at an offsite location, such as in a laboratory setting.

The method 400 includes monitoring one or more properties with the fracturing fluid monitoring system 408. Monitoring the one or more properties of the fracturing fluid may include collecting in-line spectroscopic data from the fracturing fluid flowing in a fluid transport line or other well fracturing system equipment of the modified well fracturing system. In such embodiments, monitoring the fracturing fluid may also include monitoring one or more rheological properties of the fracturing fluid.

In one or more embodiments, the spectroscopic probe collects spectral data from the fracturing fluid flowing in a fluid transport line or other well fracturing system equipment. The computer may receive, store, and process the collected in-line data to determine one or more properties of a fracturing fluid. For example, the computer may receive transmitted spectral data from the spectroscopic probe. The computer may then store and process the collected spectral data to determine one or more rheological properties of the fracturing fluid. In such embodiments, the computer processes the spectral data via comparing the spectral data to a predictive model to determine one or more rheological properties of the fracturing fluid.

The one or more determined rheological properties may be compared to a target property range of the fracturing fluid. Instances in which the one or more determined rheological properties are outside of the target property range, the method of one or more embodiments may include adjusting the fracturing fluid composition. In some embodiments, the predictive model may predict adjustments to the fracturing fluid composition to obtain a target rheological property. For example, adjustments (e.g., increases or decreases) to one or more fracturing fluid components may be generated to achieve a target rheological property. In such embodiments, the fracturing fluid composition is predicted similar to a predicted viscosity.

The fracturing fluid may be monitored as it flows through a reservoir fracturing system as described above. In one or more embodiments, the monitoring process is a continuous process during the pumping of the fracturing fluids. The fracturing fluid may be monitored for one or more properties of a fracturing fluid by the monitoring system. The one or more monitored properties of a fracturing fluid may include a viscosity of the fluid, an additive concentration, a density of the fluid, or combinations thereof. In one or more embodiments, the one or more rheological properties determined by the computer is outside of the target property range, the method of one or more embodiments includes using the computer to alter the composition of the fracturing fluid to provide a fracturing fluid within a target property range. In such embodiments, the computer system may generate and execute commands to control (e.g., open, close, or partially open) one or more valves to fracturing fluid component sources to alter the fracturing fluid composition.

When the collected spectral data from the spectroscopic probe of one or more embodiments does not correspond with the fracturing fluid predictive model, the computer may be used to control one or more fracturing equipment components in the well fracturing system to alter the fracturing fluid as described above.

Embodiments of the present disclosure may provide at least one of the following advantages. The systems and methods are applicable for sites in which reservoir fracturing occurs. In one or more embodiments, the systems and methods can classify fracturing fluids as a function of spectroscopic measurements to provide in-line viscosity values of the fracturing fluids. Embodiments of the present disclosure may also provide faster, safer and more cost-effective system and method to avoiding manual sampling of fracturing fluid and benchtop viscosity measurements that may not be fully representative of the bulk fluid data. The correlations generated by systems and methods of the present disclosure may provide information about fracturing fluids without performing the customary extensive and time-consuming fracturing fluid assays to provide a step toward quality assurance and quality control automation.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A monitoring system comprising:
a spectroscopic probe in fluid connection with a fracturing fluid;
reference spectra obtained from one or more fracturing fluid components; and
a computer in communication with the spectroscopic probe,
wherein the computer comprises a predictive model generated from the reference spectra, and
wherein the computer includes instructions to compare spectroscopic data obtained from the spectroscopic probe with the predictive model.

2. The monitoring system of claim 1, wherein the computer is configured to generate one or more reference data sets from the reference spectra.

3. The monitoring system of claim 2, wherein the computer includes instructions to build a predictive model from the one or more reference data sets.

4. The monitoring system of claim 1, wherein the fracturing fluid is a continuously flowed fracturing fluid.

5. The monitoring system of claim 1, wherein the reference spectra are obtained from a plurality of component reference fluids.

6. The monitoring system of claim 1, wherein the spectroscopic probe collects spectroscopic data in an infrared wavelength range.

7. The monitoring system of claim 1, wherein the spectroscopic probe is a Fourier Transform Infrared Spectroscopic (FTIR) probe, a Dual Comb Spectroscopic (DCS) probe, a Multi Comb Spectroscopic probe, a wavelength specific probe, or combinations thereof.

8. The monitoring system of claim 7, wherein the wavelength specific probe collects spectroscopic measurements in an infrared range selected from the group consisting of a near infrared (NIR) wavelength range, a mid-infrared (MIR) wavelength range, a far infrared (FIR) wavelength range, or combinations thereof.

9. A modified well fracturing system comprising:
a monitoring system located at a surface location of a hydrocarbon-bearing reservoir, wherein the monitoring system is in fluid connection with a fracturing fluid source, the monitoring system comprising:
a spectroscopic probe in fluid connection with a fracturing fluid;
reference spectra obtained from one or more fracturing fluid components; and
a computer in communication with the spectroscopic probe, wherein the computer is configured to compare spectroscopic data obtained from the spectroscopic probe with a predictive model generated from the reference spectra; and
a surface pump in fluid connection with the fracturing fluid source and a wellbore of the hydrocarbon-bearing reservoir.

10. The modified well fracturing system of claim 9, wherein the monitoring system is configured to collect spectroscopic data selected from the group consisting of Fourier Transform Infrared Spectroscopic (FTIR) data, a Dual Comb Spectroscopic (DCS) data, Multi Comb Spectroscopic data, wavelength specific data, or combinations thereof.

11. The modified well fracturing system of claim 10, wherein the wavelength specific data comprises spectroscopic measurements in an infrared wavelength range selected from the group consisting of a near infrared (NIR) wavelength range, a mid-infrared (MIR) wavelength range, a far infrared (FIR) wavelength range, or combinations thereof.

12. The modified well fracturing system of claim 9, wherein the spectroscopic probe is positioned between the surface pump and the fracturing fluid source.

13. A method for monitoring a fracturing fluid, the method comprising:
obtaining reference spectra for one or more fracturing fluid components to generate one or more data sets;
building a predictive model with the one or more data sets to assign rheological properties to different fracturing fluid compositions;
installing a monitoring system on fracturing equipment to provide a modified well fracturing system, the monitoring system comprising:
a spectroscopic probe in fluid connection with the fracturing fluid; and
a computer in communication with the spectroscopic probe, wherein the computer is configured to build the predictive model, compare measured spectroscopic data from the spectroscopic probe, or combinations thereof, and
injecting the fracturing fluid through the modified well fracturing system; and
collecting spectral data of the fracturing fluid.

14. The method of claim 13, further comprising building the predictive model with chemometric analysis on the one or more data sets generated from the reference spectra.

15. The method of claim 13, further comprising building the predictive model with one or more machine learning algorithms and the one or more data sets generated from the reference spectra.

16. The method of claim 13, further comprising:
comparing spectral data collected from the fracturing fluid to the predictive model to determine one or more rheological properties of the fracturing fluid.

17. The method of claim 16, further comprising:
when the one or more rheological properties derived from the predictive model is outside of a target property range, altering the composition of the fracturing fluid.

18. The method of claim 13, wherein the reference data, the spectral data, or both comprise data selected from the group consisting of Fourier Transform Infrared Spectroscopy (FTIR), a Dual Comb Spectroscopy (DCS), Multi Comb Spectroscopy, wavelength specific spectroscopy, or combinations thereof.

19. The method of claim 13, wherein obtaining reference data for one or more fracturing fluid components comprises obtaining reference data for a plurality of concentrations of one of more fracturing fluid components.

20. The method of claim 13, further comprising processing the obtained reference data to one or more data sets with the computer of the monitoring system.
